(19) United States
(12) Patent Application Publication
(10) Pub. No.: US 2024/0266047 A1
(43) Pub. Date: Aug. 8, 2024

(54) MEDICAL INFORMATION PROCESSING SYSTEM, MEDICAL INFORMATION PROCESSING METHOD, AND RECORDING MEDIUM

(71) Applicant: CANON MEDICAL SYSTEMS CORPORATION, Tochigi (JP)

(72) Inventors: Michitaka SUGAWARA, Utsunomiya (JP); Mariko SHIBATA, Nasushiobara (JP); Kohei WATANABE, Utsunomiya (JP)

(73) Assignee: CANON MEDICAL SYSTEMS CORPORATION, Tochigi (JP)

(21) Appl. No.: 18/401,799

(22) Filed: Jan. 2, 2024

(30) Foreign Application Priority Data
Feb. 6, 2023 (JP) 2023-016138

(51) Int. Cl. G16H 50/20; G16H 10/20; G16H 30/20

(52) U.S. Cl. CPC G16H 50/20; G16H 10/20; G16H 30/20

(57) ABSTRACT

A medical information processing system according to an embodiment of the present disclosure includes processing circuitry. The processing circuitry is configured to detect an abnormal site of a subject on the basis of an image of the subject. The processing circuitry is configured to extract a disease candidate on the basis of the abnormal site. The processing circuitry is configured to specify a query for performing a diagnosing process on the disease candidate. The processing circuitry is configured to present the query. With this configuration, the medical information processing system according to the present embodiment is able to assist medical doctors in making proper diagnoses.

### CROSS-REFERENCE TO RELATED APPLICATIONS

This application is based upon and claims the benefit of priority from Japanese Patent Application No. 2023-016138, filed on Feb. 6, 2023; the entire contents of which are incorporated herein by reference.

### FIELD

Embodiments described herein relate generally to a medical information processing system, a medical information processing method, and a recording medium.

### BACKGROUND

In medical question-and-answer (hereinafter “Q and A”) processes in recent years, for example, a medical doctor may assess symptoms of a patient and ask the patient questions or may ask the patient questions in advance through a dedicated terminal so as to determine optimal queries (Q and A items) on the basis of a response result from the patient. In some Q and A processes, patients may not be able to respond about a minor pain of which the patients themselves are not aware. For example, there is a possibility that proper timing for treatment may be missed, when signs of minor pains of which the patients themselves are not aware are overlooked.

### BRIEF DESCRIPTION OF THE DRAWINGS

FIG. 1 is a block diagram illustrating an exemplary configuration of a medical information processing system according to a first embodiment;

FIG. 2 is a diagram illustrating an exemplary configuration of a medical information processing apparatus in the medical information processing system according to the first embodiment;

FIG. 3 is a diagram illustrating an application example at a hospital regarding the medical information processing system according to the first embodiment;

FIG. 4 is a conceptual diagram depicting a situation where a patient image is acquired, regarding the medical information processing system according to the first embodiment;

FIG. 5 is a schematic diagram illustrating processes performed at the hospital regarding the medical information processing system according to the first embodiment;

FIG. 6 is a drawing for explaining a scene analysis performed in the medical information processing system according to the first embodiment;

FIG. 7 is a flowchart illustrating processes performed by the medical information processing system according to the first embodiment;

FIG. 8A is a table illustrating an example of a process of speculating a disease candidate from an abnormal site, in the medical information processing system according to the first embodiment;

FIG. 8B is a diagram illustrating another example of the process of speculating a disease candidate from an abnormal site, in the medical information processing system according to the first embodiment;

FIG. 9 is a drawing illustrating an example of a questionnaire for a new patient, used in the medical information processing system according to the first embodiment;

FIG. 10 is a drawing illustrating an example of a query to an outpatient used in the medical information processing system according to the first embodiment;

FIG. 11 is a drawing illustrating another example of a query to an outpatient used in the medical information processing system according to the first embodiment;

FIG. 12 is a drawing illustrating yet another example of a query to an outpatient used in the medical information processing system according to the first embodiment;

FIG. 13 is a drawing illustrating another example of a questionnaire used in the medical information processing system according to the first embodiment;

FIG. 14 is a schematic diagram in which images of a patient are compared in a time series, in a medical information processing system according to a second embodiment;

FIG. 15 is a drawing for explaining a modification example of the medical information processing systems of the present embodiments;

FIG. 16 is a drawing for explaining another modification example of the medical information processing systems of the present embodiments;

FIG. 17A is a drawing for explaining yet another modification example of the medical information processing systems of the present embodiments; and

FIG. 17B is a drawing for explaining yet another modification example of the medical information processing systems of the present embodiments.

### DETAILED DESCRIPTION

A medical information processing system according to an embodiment of the present disclosure includes processing circuitry. The processing circuitry is configured to detect an abnormal site of a subject on the basis of an image of the subject. The processing circuitry is configured to extract a disease candidate on the basis of the abnormal site. The processing circuitry is configured to specify a query for performing a diagnosing process on the disease candidate. The processing circuitry is configured to present the query.

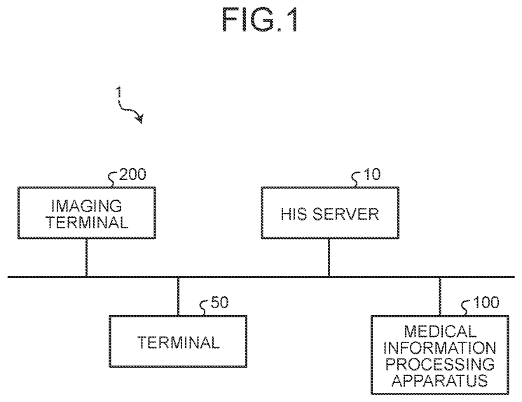

Exemplary embodiments of a medical information processing system, a medical information processing method, and a recording medium will be explained in detail, with reference to the accompanying drawings. Although the medical information processing system illustrated in FIG. 1 is depicted as having one of each of the apparatuses, the system in actuality may further include two or more apparatuses.

### First Embodiment

FIG. 1 is a block diagram illustrating an exemplary configuration of a medical information processing system 1 according to a first embodiment. The medical information processing system 1 illustrated in FIG. 1 is a system including a Hospital Information System (HIS), a radiology department information management system called a Radiology Information System (RIS), a medical image management system called a Picture Archiving and Communication System (PACS), and an electronic medical record system. The medical information processing system 1 includes an HIS server 10, a terminal 50, a medical information processing apparatus 100, and imaging terminals 200.

The HIS server 10, the terminal 50, the medical information processing apparatus 100, and the imaging terminals 200 are connected, for example, to an intra-hospital Local Area Network (LAN) installed in a hospital and are configured to transmit information to prescribed apparatuses and to receive information transmitted thereto from prescribed apparatuses. Further, the HIS server 10 may be connected to an external network, in addition to the intra-hospital LAN.

For example, the terminal 50 is used by a user engaged in diagnosis/treatment processes for patients. For example, the user may be a medical provider such as a medical doctor or a nurse. Examples of the terminal 50 include a personal Computer (PC), a tablet-type PC, a Personal Digital Assistant (PDA), and a mobile terminal.

The electronic medical record system is configured to create an electronic medical record that has recorded therein patient information such as prescriptions provided for a patient, nurse records, and medical examinations and to store the created electronic medical record into storage circuitry within the system. For example, the HIS server 10 and the medical information processing apparatus 100 are incorporated in the electronic medical record system.

The HIS server 10 includes a medical database configured to manage information generated in the hospital. Examples of the information generated in the hospital include the patient information. Further, the medical database may be connected to an external network such as a cloud.

The patient information includes basic information of the patient (patient basic information) and hospitalization information. The patient basic information includes a patient ID, the family and given names, the date of birth, the gender, the blood type, a height, a weight, and the like. As the patient ID, identification information uniquely identifying the patient is set. The hospitalization information of the patient includes information indicating numerical values (measurement values), diagnosis/treatment records, and the like, as well as information indicating the recording dates/times thereof. For instance, examples of the hospitalization information of the patient include nurse records kept by nurses and information about medical examinations ordered from examination departments, meal arrangements during hospitalization, and the like. For example, prescriptions are recorded by medical doctors into the electronic medical record. The nurse records are recorded by the nurses into the electronic medical record.

The imaging terminals 200 each include a camera. The two or more imaging terminals 200 are provided in the hospital. For example, the imaging terminals 200 are provided inside the hospital, in certain areas such as an entrance, a reception, an accounting department, a consultation room, and a waiting room, as well as in a hallway, a staircase, and/or the like.

The medical information processing apparatus 100 illustrated in FIG. 1 is a workstation configured to cause a display of the terminal 50 to display diagnosis/treatment information, medical images, and the like of the patient, as information related to diagnosis/treatment processes for the patient.

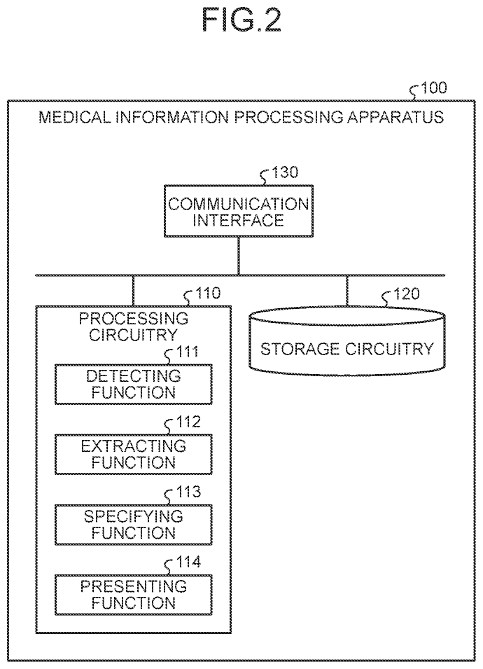

Next, details of the medical information processing apparatus 100 will be explained. FIG. 2 is a diagram illustrating an exemplary configuration of the medical information processing apparatus 100 in the medical information processing system 1 according to the first embodiment. As illustrated in FIG. 2, the medical information processing apparatus 100 includes processing circuitry 110, storage circuitry 120, and a communication interface 130.

The storage circuitry 120 is connected to the processing circuitry 110 and is configured to store therein various types of information. More specifically, the storage circuitry 120 is configured to store therein the patient information received from various systems. For example, the storage circuitry 120 is realized by using a semiconductor memory element such as a Random Access Memory (RAM) or a flash memory, or a hard disk, an optical disc, or the like. In this situation, the storage circuitry 120 is an example of a storage unit. For example, the communication interface 130 is a Network Interface Card (NIC) or the like and is configured to communicate with other apparatuses.

The processing circuitry 110 is configured to control constituent elements of the medical information processing apparatus 100. For example, as illustrated in FIG. 2, the processing circuitry 110 is configured to execute a detecting function 111, an extracting function 112, a specifying function 113, and a presenting function 114. In this situation, for example, processing functions performed by the constituent elements of the processing circuitry 110, namely, the detecting function 111, the extracting function 112, the specifying function 113, and the presenting function 114, are recorded in the storage circuitry 120 in the form of computer-executable programs. The processing circuitry 110 is a processor configured to realize the functions corresponding to the programs, by reading and executing the programs from the storage circuitry 120. In other words, the processing circuitry 110 that has read the programs has the functions illustrated within the processing circuitry 110 in FIG. 2.

The term “processor” used in the above explanations may denote, for example, circuitry such as a Central Processing Unit (CPU), a Graphics Processing Unit (GPU), or an Application Specific Integrated Circuit (ASIC). In another example, the term “processor” may denote circuitry such as a programmable logic device. Examples of the programmable logic device include a Simple Programmable Logic Device (SPLD) and a Complex Programmable Logic Device (CPLD). Other examples of the programmable logic device include a field programmable gate array (FPGA). When the processor is a CPU, for example, the processor is configured to realize the functions by reading and executing the programs saved in the storage circuitry 120. In contrast, when the processor is an ASIC, for example, instead of having the programs saved in the storage circuitry 120, the programs are directly incorporated in the circuitry of the processor. Further, the processors in the present embodiments do not each necessarily have to be structured as a single piece of circuitry. It is also acceptable to structure one processor by combining together a plurality of pieces of independent circuitry so as to realize the functions thereof. Furthermore, it is also acceptable to integrate two or more of the constituent elements illustrated in FIG. 2 into a single processor so as to realize the functions thereof.

Alternatively, the detecting function 111, the extracting function 112, the specifying function 113, and the presenting function 114 of the medical information processing apparatus 100 may be provided in mutually-different apparatuses. In that situation, as the functions of the medical information processing apparatus 100, the medical information processing system 1 has the apparatus provided with the detecting function 111, the apparatus provided with the extracting function 112, the apparatus provided with the specifying function 113, and the apparatus provided with the presenting function 114.

In medical Q and A processes in recent years, for example, a medical doctor may assess symptoms of a patient and ask the patient questions or may ask the patient questions in advance through a dedicated terminal using an Artificial Intelligence (AI) algorithm for deep learning or the like, so as to determine optimal queries (Q and A items) on the basis of a response result from the patient. In some Q and A processes, patients may not be able to respond about a minor pain of which the patients themselves are not aware. For example, when a patient has a chronic disease, there may be a sign of a minor pain of which the patient himself/herself is not aware, such as a referred pain occurring from the chronic disease. For example, when signs of minor pains of which the patients themselves are not aware are overlooked, there is a possibility that proper timing for treatment may be missed, for heart diseases, liver failure, or failure of other internal organs such as the gallbladder. It is therefore necessary to provide a lead to proper treatment, by finding signs of minor pains of which even the patients themselves are not aware.

Thus, the medical information processing system 1 according to the present embodiment is configured to perform the following processes to assist medical doctors in making proper diagnoses. To begin with, in the medical information processing system 1 according to the present embodiment, the detecting function 111 is configured to detect an abnormal site of a subject (hereinafter, “patient”) on the basis of an image of the patient. The extracting function 112 is configured to extract a disease candidate on the basis of the abnormal site. The specifying function 113 is configured to specify a query for performing a diagnosing process on the disease candidate. The presenting function 114 is configured to present the query. In this situation, the detecting function 111, the extracting function 112, the specifying function 113, and the presenting function 114 are examples of a detecting unit, an extracting unit, a specifying unit, and a presenting unit, respectively.

Functions of the medical information processing system 1 according to the present embodiment will be explained.

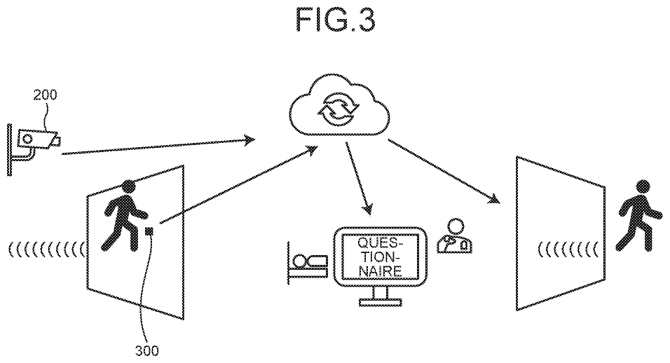

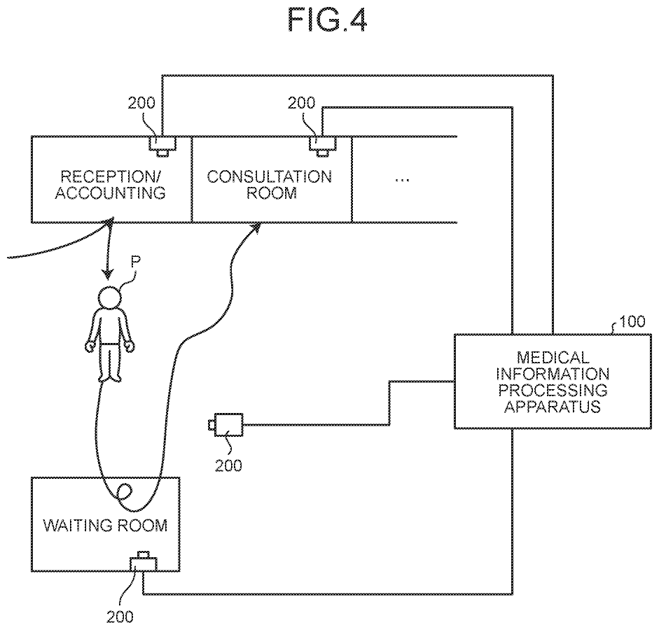

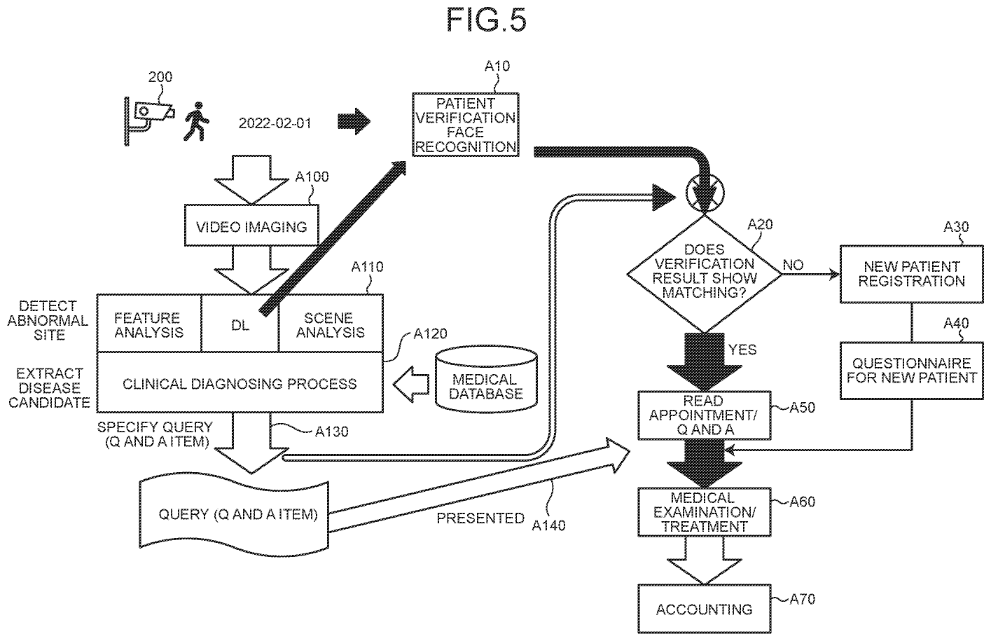

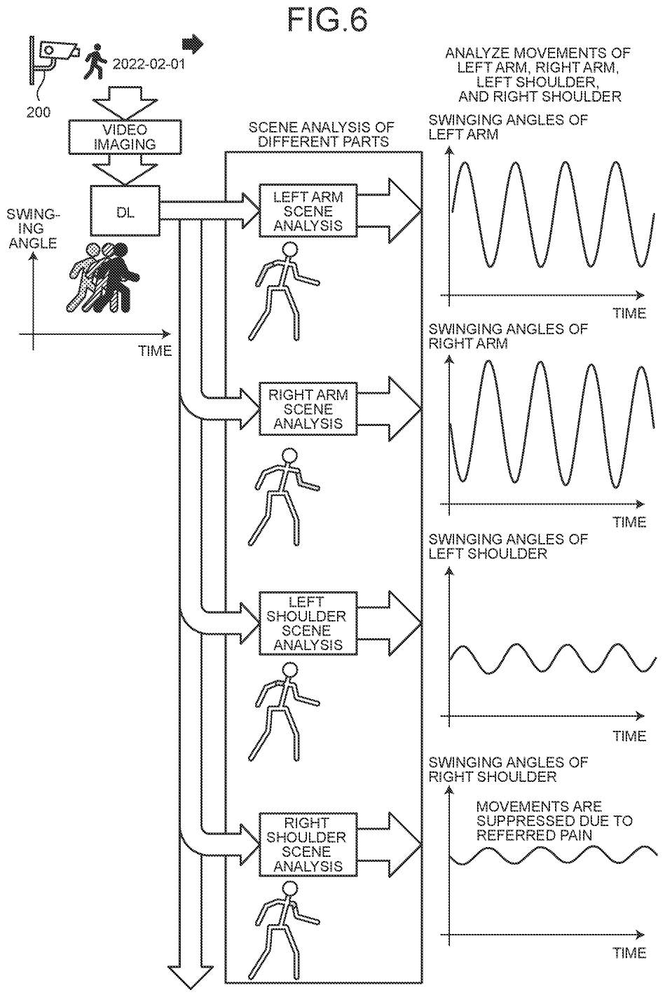

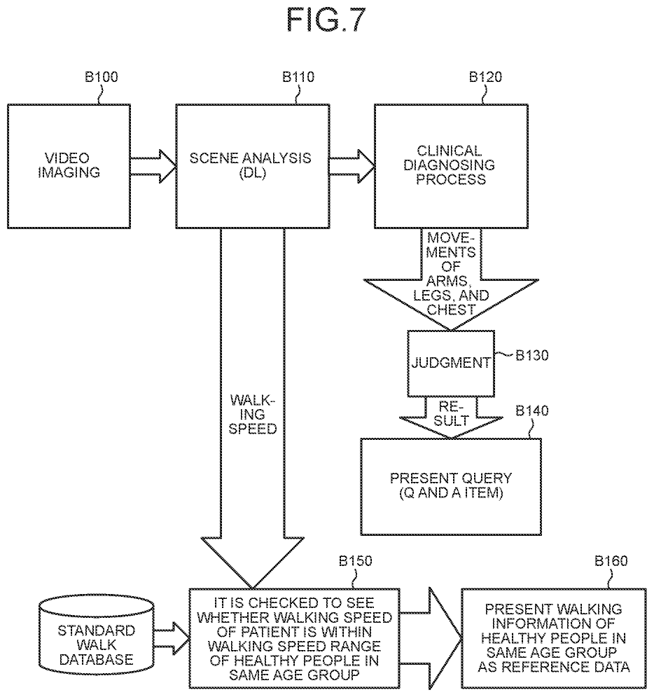

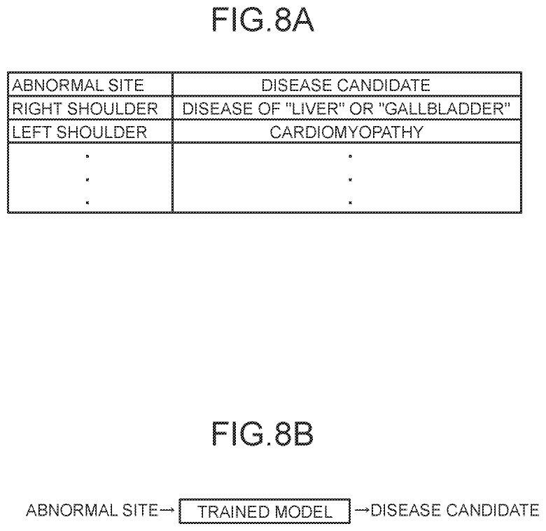

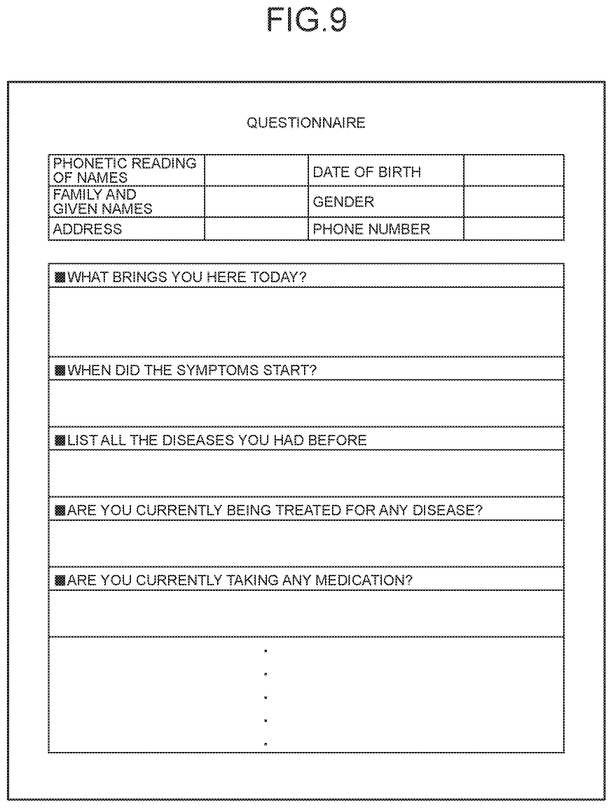

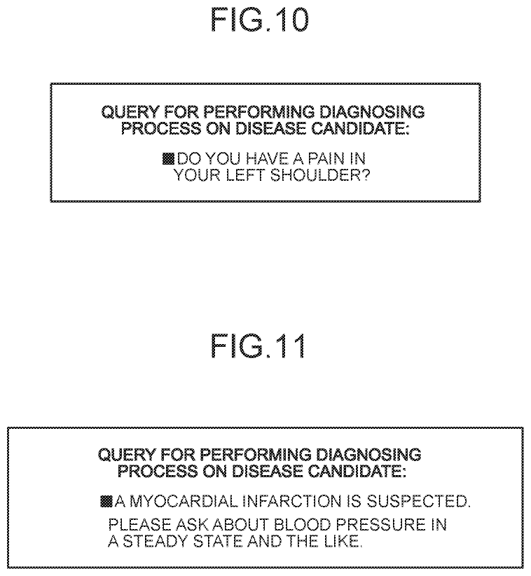

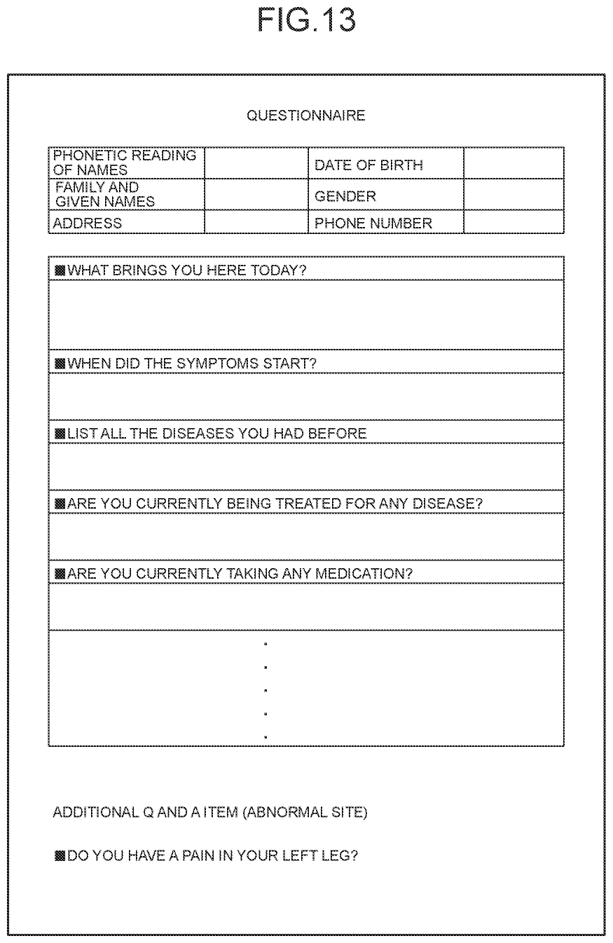

FIG. 3 is a diagram illustrating an application example at a hospital regarding the medical information processing system 1 according to the first embodiment. FIG. 4 is a conceptual diagram depicting a situation where a patient image is acquired. FIG. 5 is a schematic diagram illustrating processes performed at a hospital regarding the medical information processing system 1 according to the first embodiment. FIG. 6 is a drawing for explaining a scene analysis performed in the medical information processing system 1 according to the first embodiment. FIG. 7 is a flowchart illustrating processes performed by the medical information processing system 1 according to the first embodiment. FIGS. 8A and 8B are drawings illustrating examples of the process of speculating a disease candidate from an abnormal site. FIG. 9 is a drawing illustrating an example of a questionnaire for a new patient. FIGS. 10, 11, and 12 are drawings illustrating examples of queries to an outpatient. FIG. 13 is a drawing illustrating another example of a questionnaire.

FIG. 3 depicts a manner in which a subject (a patient) visits to a hospital. In the medical information processing system 1 according to the first embodiment, from the time when the patient arrives at the hospital until the patient finishes a diagnosis/treatment process and exits a consultation room, the imaging terminals 200 are configured to image situations surrounding the patient.

In the present example, it is assumed that the medical database is provided in a cloud. In the medical database, the image of the patient visiting the hospital is recorded in an electronic medical record while being kept in correspondence with his/her patient information. For example, at the point in time when the patient goes through an entrance of the hospital, the imaging terminals 200 starts imaging the patient.

The image of the patient is an image taken at locations including at least one selected from between an entrance and a waiting room of the hospital. FIG. 4 depicts a manner in which the image of the patient is acquired. For example, at the hospital, the imaging terminals 200 each including a camera are installed in certain areas such as an entrance, a reception, an accounting department, a consultation room, and a waiting room, as well as in a hallway, a staircase, and/or the like. For example, the imaging terminals 200 are configured to image a patient P who went through the entrance of the hospital, the reception, and the waiting room, and arrived at the consultation room and are configured to transmit video files containing the image of the patient P to the medical information processing apparatus 100. For example, the processing circuitry 110 of the medical information processing apparatus 100 is configured to store the video files of the patient P transmitted thereto from the imaging terminals 200 into the medical database provided in the cloud. Alternatively, the imaging terminals 200 may store the video files of the patient P into the medical database in the cloud, without the intermediation of the medical information processing apparatus 100.

For example, when the patient comes to the hospital on “Feb. 1, 2022”, the imaging terminals 200 images the patient through video imaging (step A100 in FIG. 5; step B100 in FIG. 7).

In that situation, by using an AI algorithm for Deep Learning (DL) or the like, for example, the detecting function 111 is configured to compare the image of the patient taken by the imaging terminals 200 with an image of the patient in the medical database, as a face recognition process. Further, as a patient verification process, the HIS server 10 compares information such as the family and given names, the date of birth, the gender, and/or the like of the patient recorded on a card 300 presented by the patient at the reception of the hospital, with information such as the family and given names, the date of birth, the gender, and/or the like of the patient included in the patient information within the medical database (step A10 in FIG. 5). Examples of the card 300 include a health insurance certificate, a consultation card, and a social security number card.

Processes at the time of verifying the patient and performing the face recognition process will be explained later.

In the present example, when the imaging terminals 200 have imaged the patient through the video imaging, the detecting function 111, the extracting function 112, and the specifying function 113 are configured to analyze a relationship between the image of the patient and a chronic disease of the patient, by using an AI algorithm for Deep Learning (DL) or the like.

More specifically, from the image of the patient, the detecting function 111 is configured to detect the abnormal site of the patient by performing a feature analysis and a scene analysis. For example, by performing the feature analysis and the scene analysis, the detecting function 111 is configured to detect, as the abnormal site of the patient, a site of the patient's body having unsatisfactory movements in comparison to healthy movements (step A110 in FIG. 5; step B110 in FIG. 7).

The feature analysis and the scene analysis will be explained. When patients have a pain in their bodies, because the site causing a pain makes an unsatisfactory movement, patients tend to use their bodies so as not to move such a site either consciously or unconsciously. In the present embodiment, by imaging unconscious body movements of the patient at the time of the hospital visit and performing the image analysis, it is possible to detect a symptom of which the patient is not aware.

To begin with, as the feature analysis, the detecting function 111 is configured to read the video files of the patient from the medical database and to extract features of the patient through Deep Learning (DL) or the like from frames (still images) in the read video files of the patient, so as to convert the image into segmentable data.

Subsequently, as the scene analysis, the detecting function 111 is configured to analyze, through a segmentation process, sites such as the head, the four limbs, the femurs, the foot bones, the tibiae, the fibulae, the collarbones, the humeri, the ulnae, the radii, and the hand bones, from the frames (the still images) in the video files of the patient, for example. After that, the detecting function 111 is configured to record positions and movements of the sites of the patient into the medical database as a motion capture process, and to evaluate the movements of each of the sites during walking of the patient. For example, changes in the movements of the sites and walking speeds are influenced by the strength of a referred pain. As a change in the movements of the sites, the detecting function 111 is configured to find a point exhibiting a large standard deviation (SD) value, for example, and to calculate a correlation between movement change points caused by the referred pain and the walking speed, and is thus able to determine whether or not the patient has unsatisfactory movements due to the referred pain, in comparison to healthy movements.

FIG. 6 depicts a manner in which the scene analysis is performed on the movements of the patient. For example, the movements of the sites such as the head, the left arm, the right arm, the left shoulder, the right shoulder, the hip (left), the hip (right), the left knee, the right knee, the right foot (ankle), the left foot (ankle), and/or the like of the patient cyclically change during walking. In the example in FIG. 6, the detecting function 111 is configured to perform the scene analysis on the movements of the sites such as the left arm, the right arm, the left shoulder, and the right shoulder of the patient. In FIG. 6, the horizontal axis expresses time during walking of the patient, whereas the vertical axis expresses swinging angles of the left arm, the right arm, the left shoulder, and the right shoulder during the walking of the patient. As the changes in the movements of the patient, the detecting function 111 is configured to calculate an average value and an SD value of the walking speeds of the patient, as well as a maximum value, a minimum value, and an SD value of the swinging angles of each of the sites such as the left arm, the right arm, the left shoulder, and the right shoulder of the patient, by performing the scene analysis.

It is considered that an average value of walking speeds indicates a degree of a person's health. For example, when a patient has a pain at a certain site, the patient may unconsciously favor the painful site, or activities of the patient may be hindered. As a result, the maximum value, the minimum value, and the SD value of the swinging angles of such a site are different. Accordingly, on the basis of the video files of the patient, the detecting function 111 is configured to check to see whether there is a difference between the left and the right, in the movements of the patient. In the example in FIG. 6, the maximum value, the minimum value, and the SD value of the swinging angles of the right shoulder of the patient are smaller than the maximum value, the minimum value, and the SD value of the swinging angles of the left shoulder of the patient. In that situation, there is a possibility that the patient may be feeling, in the right shoulder, a pain or a certain abnormality.

Further, by referring to a standard walk database illustrated in FIG. 7, the detecting function 111 is configured to check to see whether or not the walking speed of the patient is within a walking speed range of healthy people in the same age group as the patient, on the basis of the video files of the patient (step B150 in FIG. 7). For example, the standard walk database stores therein, in correspondence with ages, walking information related to walking of healthy people, such as walking speeds of healthy people. For example, the standard walk database may be provided in a cloud. Alternatively, the standard walk database may be provided in the storage circuitry 120 of the medical information processing apparatus 100. When the patient is feeling a referred pain or a certain abnormality, there is a possibility that the walking speed of the patient may be slower than the walking speed of healthy people in the same age group as the patient and may not be within the walking speed range of healthy people in the same age group.

Accordingly, in the example in FIG. 6, by performing the feature analysis and the scene analysis, the detecting function 111 is configured to detect the right shoulder of the patient as an abnormal site of the patient being a site of the patient's body having unsatisfactory movements in comparison to movements of healthy people in the same age group.

Further, in the example in FIG. 6, the detecting function 111 is configured to detect the abnormal site of the patient, by performing the feature analysis and the scene analysis so as to calculate the average value and the SD value of the walking speeds of the patient and the maximum value, the minimum value, and the SD value of the swinging angles of each of the sites of the patient, as the changes in the movements of the patient. However, possible embodiments are not limited to this example. For instance, as another method for detecting an abnormal site of the patient, it is also acceptable to detect the abnormal site of the patient by performing a numerical value analysis on the image of the patient, by using Hotelling's T2 method.

After that, as a clinical diagnosing process, the extracting function 112 is configured to extract a disease candidate, on the basis of the detected abnormal site (step A120 in FIG. 5; step B120 in FIG. 7). FIGS. 8A and 8B illustrate examples of the process of speculating the disease candidate from the abnormal site. When extracting the disease candidate on the basis of the abnormal site, the extracting function 112 may refer to a table keeping abnormal sites in correspondence with relevant disease candidates, as illustrated in FIG. 8A, for example, so as to speculate the disease candidate from the abnormal site. Alternatively, as illustrated in FIG. 8B, the extracting function 112 may use a trained model that has been trained through machine learning or the like while using a database keeping abnormal sites in correspondence with disease diagnosis results, so as to speculate the disease candidate from the abnormal site.

For example, a referred pain felt by a patient due to a disease in the “liver” or the “gallbladder” occurs around the “right shoulder” of the patient. For example, when a patient feels a pain in the right shoulder, movements and walking of the patient may be deteriorated, and the patient may behave differently from healthy people by raising/lowering the right shoulder having a pain, rubbing the right shoulder, changing the posture, or the like. In that situation, there is a possibility that the patient's behavior may be related to the referred pain caused by a disease in the liver or the gallbladder. Accordingly, in FIGS. 8A and 8B, the extracting function 112 extracts a disease in the “liver” or the “gallbladder” as a disease candidate, on the basis of the “right shoulder” being an abnormal site of the patient.

Similarly, a referred pain felt by a patient due to “cardiomyopathy” occurs around the “left shoulder” of the patient. For example, when a patient feels a pain in the left shoulder, movements and walking of the patient may be deteriorated, and the patient may behave differently from healthy people. In that situation, there is a possibility that the patient's behavior may be related to the referred pain caused by cardiomyopathy. Accordingly, in FIGS. 8A and 8B, the extracting function 112 extracts “cardiomyopathy” as a disease candidate, on the basis of the “left shoulder” being an abnormal site of the patient.

Also, similarly, a referred pain felt by a patient due to a disease in the “pancreas” or a “kidney” occurs around the “back” of the patient. For example, when a patient feels a pain in the back, movements and walking of the patient may be deteriorated, and the patient may behave differently from healthy people. In that situation, there is a possibility that the patient's behavior may be related to the referred pain caused by a disease in the “pancreas” or the “kidney”. Accordingly, in FIGS. 8A and 8B, the extracting function 112 extracts a disease in the “pancreas” or a “kidney”, as a disease candidate on the basis of the “back” being an abnormal site of the patient.

After that, the specifying function 113 is configured to specify queries (Q and A items) for performing a diagnosing process on the disease candidate (step A130 in FIG. 5; step B130 in FIG. 7).

For example, when the disease candidate is a disease in the “liver” or the “gallbladder”, the specifying function 113 is configured to specify a Q and A item “Do you have a pain in your right shoulder?” as a query for performing a diagnosing process on the disease candidate. Similarly, when the disease candidate is the disease of “cardiomyopathy”, the specifying function 113 is configured to specify a Q and A item “Do you have a pain in your left shoulder?” as a query for performing a diagnosing process on the disease candidate. Also, similarly, when the disease candidate is a disease in the “pancreas” or a “kidney”, the specifying function 113 is configured to specify a Q and A item “Do you have a pain in your back?” as a query for performing a diagnosing process on the disease candidate.

Next, processes performed at the time of verifying the patient and performing the face recognition will be explained.

For example, as a result of the patient verification process and the face recognition verification process, the image of the patient taken by the imaging terminals 200 may not match the image of the patient in the medical database. In another example, the information recorded on the card 300 brought by the patient such as the family and given names, the date of birth, the gender and/or the like of the patient may not match the information included in the patient information within the medical database, such as the family and given names, the date of birth, the gender, and/or the like of the patient (step A20: No in FIG. 5).

In that situation, because the patient is a new patient, a new patient registration is carried out at the reception in the hospital, so that the HIS server 10 stores information such as the family and given names, the date of birth, the gender, and/or the like of the new patient into the medical database, as patient information (step A30 in FIG. 5). Further, a questionnaire for new patients is presented to the new patient at the reception of the hospital, and the new patient is asked to fill out the questionnaire (step A40 in FIG. 5). FIG. 9 illustrates an example of the questionnaire for new patients. In the questionnaire in FIG. 9, the new patient is asked to write information such as the family and give names, the date of birth, the gender, and/or the like as personal information of the new patient. In addition, the new patient is asked to write responses to questions such as “What brings you here today?” or “When did the symptom start?”. For example, at the reception in the hospital, the HIS server 10 displays a screen presenting the questionnaire on a terminal at the reception. After that, in a consultation room in the hospital, a medical doctor performs a medical examination and/or treatment for the patient on the basis of the responses of the patient in the questionnaire (step A60 in FIG. 5). On such occasion, the medical doctor causes the electronic medical record of the patient to be read from the medical database by using the terminal 50 and records the responses of the patient and results of the medical examination and/or the treatment into the read electronic medical record. After that, the patient makes a payment or the like at the accounting department in the hospital and makes an appointment if another visit to the hospital is necessary (step A70 in FIG. 5).

For example, as a result of the patient verification process and the face recognition verification process, a verification result may indicate that the image of the patient taken by the imaging terminals 200 matches the image of the patient in the medical database. Also, a verification result may indicate that the information recorded on the card 300 of the patient such as the family and given names, the date of birth, the gender, and/or the like of the patient matches the information included in the patient information within the medical database, such as the family and given names, the date of birth, the gender, and/or the like of the patient (step A20: Yes in FIG. 5).

For example, let us assume that the patient is an outpatient, has previously filled out the questionnaire, and has an appointment. In that situation, in a consultation room in the hospital, the presenting function 114 is configured to read the electronic medical record of the patient having the appointment from the medical database (step A50 in FIG. 5) and to present the specified queries to a medical doctor, in the form of a questionnaire (step A140 in FIG. 5; step B140 in FIG. 7).

FIGS. 10 to 12 illustrate examples of the queries to an outpatient. For example, as illustrated in FIG. 10, the presenting function 114 causes the terminal 50 of the medical doctor to display a screen indicating a query. In the example of FIG. 10, the disease candidate is the disease of “cardiomyopathy”, and the screen displays a query such as “Do you have a pain in your left shoulder?” as a query for performing a diagnosing process on the disease candidate.

Further, the presenting function 114 may present a query for performing a clinical diagnosing process on the disease candidate as an additional Q and A item. For example, when the extracting function 112 extracted “cardiomyopathy” as a disease candidate on the basis of an abnormal site “left shoulder” of the patient, the specifying function 113 may specify a Q and A item “A myocardial infarction is suspected based on the abnormal site. Please ask about blood pressure in a steady state and the like”, as a query for performing a diagnosing process on the disease candidate. In that situation, as illustrated in FIG. 11, the presenting function 114 is configured to present the query such as “A myocardial infarction is suspected based on the abnormal site. Please ask about blood pressure in a steady state and the like.”, as the query for performing a clinical diagnosing process on the disease candidate.

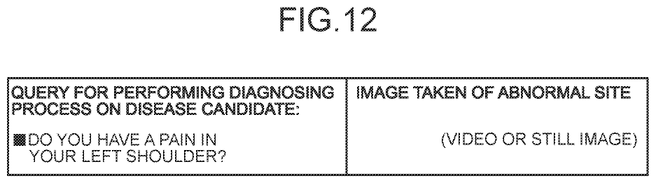

Further, when causing the terminal 50 to display the screen indicating the query “Do you have a pain in your left shoulder?” illustrated in FIG. 10, the presenting function 114 may as illustrated in FIG. 12, cause the terminal 50 to display a video or a still image taken at the time of imaging the “left shoulder” being the abnormal site.

Further, when causing the terminal 50 to display the screen indicating the query “Do you have a pain in your left shoulder?” for performing a diagnosing process on the disease candidate, the presenting function 114 may cause the terminal 50 to display information indicating that the walking speed of the patient is not within the walking speed range of healthy people in the same age group or walking information related to walking of healthy people in the same age group such as a walking speed of the healthy people, as reference data (step B160 in FIG. 7). With these arrangements, by presenting the patient with the screen displayed on the terminal 50, the medical doctor is able to present the patient with the information indicating that the walking speed of the patient is not within the walking speed range of healthy people in the same age group or the walking information related to the walking of healthy people as the reference data.

Further, in the consultation room, the medical doctor performs a medical examination and/or treatment for the patient on the basis of the responses of the patient to the queries (step A60 in FIG. 5). On such occasion, the medical doctor causes the electronic medical record of the patient to be read from the medical database by using the terminal 50 and records the responses of the patient and results of the medical examination and/or the treatment into the read electronic medical record. After that, the patient makes a payment or the like at the accounting department in the hospital and makes an appointment if another visit to the hospital is necessary (step A70 in FIG. 5).

As explained above, in the medical information processing system 1 according to the first embodiment, the symptom of which the patient is not aware undergoes the image analysis, as being triggered by the referred pain caused by the chronic disease, so that the patient is made aware through the Q and A process.

Further, the presenting function 114 may be configured to present one or more queries to the medical doctor, by adding the queries to a questionnaire to be filled out by the patient. For example, let us discuss an example in which the patient is a newly-registered patient who does not have an appointment, and the detecting function 111 detected his/her left leg as an abnormal site, at the point in time when the patient went through an entrance of the hospital. On the basis of the detected abnormal site, the extracting function 112 is configured to extract a disease candidate. The specifying function 113 is configured to specify a query for performing a diagnosing process on the disease candidate. After that, the presenting function 114 is configured to add the query to a questionnaire to be filled out by the patient and to present the questionnaire to the patient. FIG. 13 illustrates another example of the questionnaire. For example, as illustrated in FIG. 13, at the reception in the hospital, the presenting function 114 is configured to control the HIS server 10 so as to display, on a terminal at the reception, a screen having the query added to the questionnaire. On the screen illustrated in FIG. 13, a query such as “Do you have a pain in your left leg?” is displayed as the query for performing a diagnosing process on the disease candidate.

As explained above, in the medical information processing system 1 according to the first embodiment, the detecting function 111 is configured to detect the abnormal site of the patient, on the basis of the image of the patient. The extracting function 112 is configured to extract the disease candidate on the basis of the abnormal site. Further, the specifying function 113 is configured to specify the query for performing a diagnosing process on the disease candidate. The presenting function 114 is configured to present the query. For example, when the patient is an outpatient, a sign of a minor pain (e.g., a referred pain caused by a chronic disease) of which the patient himself/herself is not aware may be exhibited. For this reason, in the medical information processing system 1 according to the first embodiment, by discovering the sign of such a minor pain of which the patient himself/herself is not aware, the medical doctor is able to properly provide treatment for heart diseases, liver failure, and failure in other internal organs such as the gallbladder, on the basis of such a referred pain.

### Second Embodiment

In the first embodiment, for example, when the outpatient comes to the hospital on “Feb. 1, 2022”, the detecting function 111 is configured to detect the abnormal site of the patient on the basis of the image of the patient. The extracting function 112 is configured to extract the disease candidate on the basis of the abnormal site. The specifying function 113 is configured to specify the query for performing a diagnosing process on the disease candidate. As another example, in a second embodiment, images of the patient may be compared in a time series, so as to judge a state (improved or worsened) of the abnormal site of the patient, and to use a judgment result as a base of a query.

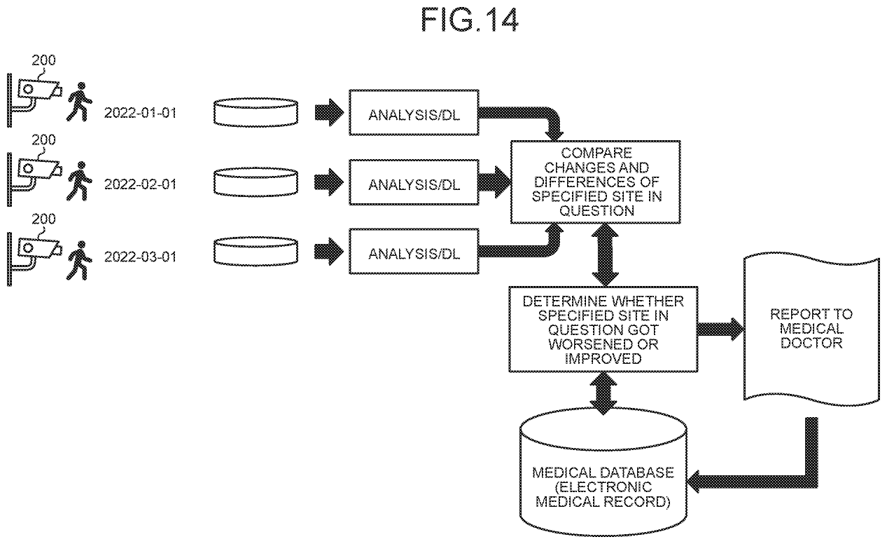

FIG. 14 is a schematic diagram in which images of a patient are compared in a time series, in the medical information processing system 1 according to the second embodiment.

For example, when the patient comes to the hospital on “Jan. 1, 2022”, “Feb. 1, 2022”, and “Mar. 1, 2022”, the imaging terminals 200 installed in the same positions image the patient, so that video files of the patient from each visit are stored into the medical database in a cloud. Subsequently, with respect to the dates “Jan. 1, 2022”, “Feb. 1, 2022”, and “Mar. 1, 2022”, the detecting function 111 is configured to perform a feature analysis and a scene analysis through Deep Learning (DL) or the like and to thereby calculate, from the images of the patient, an average value and an SD value of the walking speeds of the patient and a maximum value, a minimum value, an SD value, and/or the like of swinging angles of each of the sites of the patient, as changes in the patient's movements. After that, the detecting function 111 is configured to judge whether the state of a specified site in question got worsened or improved as compared with before, by comparing, in a time series, changes and differences in the movements of the specified site in question (the abnormal site). After that, the detecting function 111 is configured to read the electronic medical record of the patient from the medical database and to record information about the changes in the movements of the patient and a judgment result from the dates “Jan. 1, 2022”, “Feb. 1, 2022”, and “Mar. 1, 2022” into the read electronic medical record. Further, the extracting function 112 is configured to extract a disease candidate on the basis of the abnormal site. The specifying function 113 is configured to specify a query for performing a diagnosing process on the disease candidate. The presenting function 114 is configured to present the query. Further, as a report to the medical doctor, the extracting function 112, the specifying function 113, and the presenting function 114 are each configured to read the electronic medical record of the patient from the medical database and to record information about the abnormal site, the disease candidate, the query, and/or the like from the dates “Jan. 1, 2022”, “Feb. 1, 2022”, and “Mar. 1, 2022” into the read electronic medical record.

As explained herein, in the second embodiment, by comparing the images of the patient in the time series, the state (improved or got worsened) of the abnormal site of the patient is judged, so as to use the judgment result as a base of the query. The precision level of the Q and A process is therefore enhanced.

### Other Embodiments

Although a number of embodiments have thus been explained, the present disclosure may be carried out in various different modes other than the embodiments described above.

In an embodiment, the extracting function 112 may be configured to extract a disease candidate, further on the basis of a medical department with which the patient was registered at the reception. For example, when the patient is visiting a neurology department, the extracting function 112 may extract a disease candidate on the basis of an abnormal site of the patient, by narrowing down the options to neurological diseases.

Further, in the embodiment described above, when performing the scene analysis, the detecting function 111 is configured to detect the site of the patient's body having unsatisfactory movements as the abnormal site of the patient; however, possible embodiments are not limited to this example. For instance, in a modification example of the present embodiments, a scene analysis may be performed for the processes starting with the detecting function 111 detecting a site of the patient's body having unsatisfactory movements as an abnormal site of the patient, and up to the extracting function 112 subsequently extracting the disease candidate on the basis of the abnormal site.

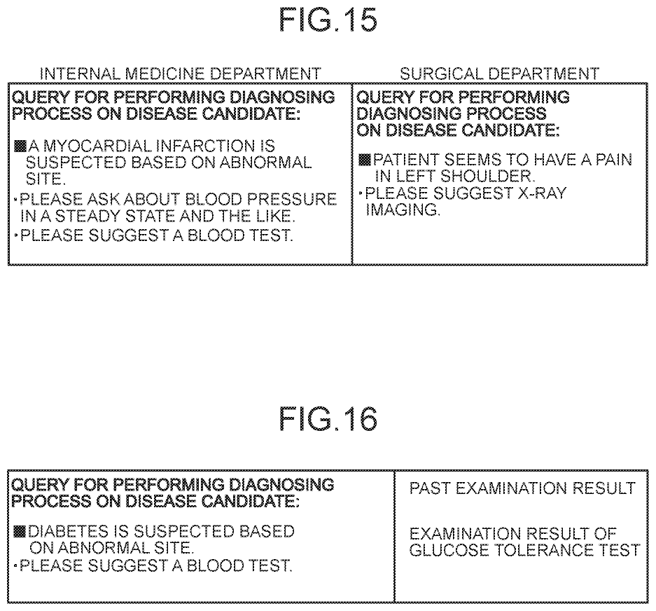

In another modification example of the present embodiments, when presenting the additional Q and A item as the query for performing a clinical diagnosing process of the disease candidate, the presenting function 114 may be configured to present queries while distinguishing a query for the medical department being visited and another query for a medical department different from the visited medical department. For example, as illustrated in FIG. 15, the presenting function 114 may cause the terminal 50 to display a query such as “A myocardial infarction is suspected based on the abnormal site. Please ask about blood pressure in a steady state and the like. Please suggest a blood test.” as a query for performing a clinical diagnosing process on the disease candidate at the internal medicine department. At the same time, the presenting function 114 may be configured to cause the terminal 50 to display a query such as “The patient seems to have a pain in the left shoulder. Please suggest X-ray imaging” as a query for performing a clinical diagnosing process on the disease candidate at the surgical department. For example, by referring to the screen displayed on the terminal 50, as the query for performing the clinical diagnosing process on the disease candidate at the internal medicine department, an internal medicine doctor informs the patient that a myocardial infarction is suspected, asks about blood pressure in a steady state and the like, and suggests a blood test. Further, as the query for performing the clinical diagnosing process on the disease candidate at the surgical department, the internal medicine doctor further asks the patient about the pain in the left shoulder, suggests X-ray imaging, and informs the patient that the internal medicine doctor will contact the surgical department about the X-ray imaging.

Further, in yet another modification example of the present embodiments, when presenting the additional Q and A item as the query for performing the clinical diagnosing process on the disease candidate, the presenting function 114 may be configured to also present, as reference information, information such as past examination results and/or clinical images of the patient, if available, that is relevant to the diagnosing process of the disease candidate. For example, as illustrated in FIG. 16, the presenting function 114 may be configured to present a query such as “Diabetes is suspected based on the abnormal site. Please suggest a blood test”, as a query for performing a clinical diagnosing process on the disease candidate. At the same time, as a past examination result of the patient relevant to performing the diagnosing process on the disease candidate, the presenting function 114 may be configured to present an examination result from a glucose tolerance test.

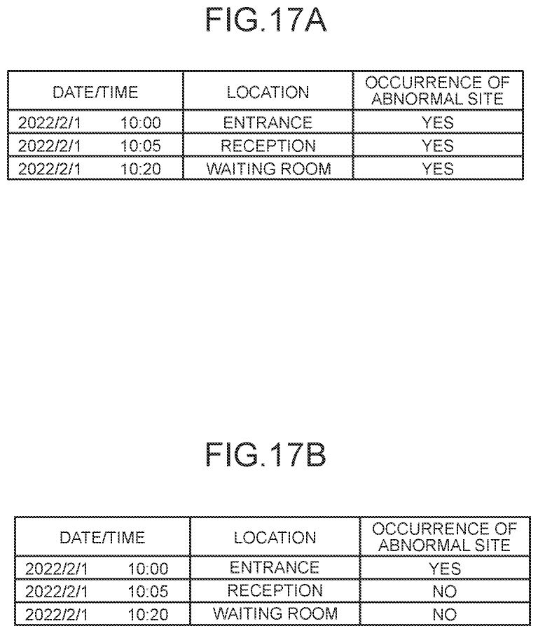

Further, in the embodiment described above, data is accumulated from the scene analysis performed on the plurality of points in time such as at the entrance, the reception, and the waiting room of the hospital. In that situation, in a modification example of the present embodiments, the detecting function 111 is able to determine, by performing the scene analysis, whether the abnormal site is occurring constantly, by detecting whether or not the abnormal site is occurring at all of the plurality of points in time. For example, as illustrated in FIG. 17A, the detecting function 111 may be configured to determine that the abnormal site is occurring constantly from a chronic disease such as diabetes, when detecting that the abnormal site is occurring at all of the plurality of points in time, such as at the entrance, the reception, and the waiting room of the hospital. In that situation, the extracting function 112 may be configured to extract the disease candidate such as diabetes on the basis of the abnormal site, so that the specifying function 113 specifies a query for performing a diagnosing process on the disease candidate. In contrast, as illustrated in FIG. 17B, when the detecting function 111 detected the occurrence of an abnormal site at an entrance of the hospital, but detected that the abnormal site is not also occurring at a plurality of other points in time such as at the reception and the waiting room of the hospital, the detecting function 111 may determine that the abnormal site accidentally occurred due to a reason such as spraining the ankle. In that situation, the extracting function 112 may be configured to extract the suspicion about spraining the ankle on the basis of the abnormal site, so that the specifying function 113 specifies a query for performing a diagnosing process on the suspected spraining of the ankle.

Further, in yet another modification example of the present embodiments, when the detecting function 111 is unable to detect, from a video of a certain point in time, a site of the patient's body having unsatisfactory movements as an abnormal site of the patient, the detecting function 111 may be configured to detect, from another video of a different point in time, a site of the patient's body having unsatisfactory movements as an abnormal site of the patient. In another example, a medical doctor may prompt the patient to make movements while there is no obstacle or belongings, so that the detecting function 111 detects, from a video, a site of the patient's body having unsatisfactory movements as an abnormal site of the patient. When a patient appears to have a pain in the left leg, a medical doctor may prompt the patient to go up and down stairs, so that the detecting function 111 detects, from a video, the site of the patient's body having unsatisfactory movements as an abnormal site of the patient.

Further, in yet another modification example of the present embodiments, captured scenes in images may be kept in correspondence in advance with detectable abnormal sites. For example, for stairs, stairs may be kept in correspondence, in advance, with the legs of patients, so that the detecting function 111 detects an abnormal site of a patient on the basis of movements of the patient's legs. For the reception, the reception may be kept in correspondence, in advance, with movements of the arms and the hands of patients, so that the detecting function 111 detects an abnormal site of a patient on the basis of movements of the patient's arms and hands.

Further, in yet another modification example of the present embodiments, when a disease or a disease candidate was extracted as a past diagnosis result, the detecting function 111 may be configured to detect an abnormal site corresponding to the disease or the disease candidate given in the past diagnosing process, so as to perform a scene analysis by extracting a captured scene from which the corresponding abnormal site is detectable.

The constituent elements of the apparatuses depicted in the drawings of the present embodiments are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Further, all or an arbitrary part of the processing functions performed by the apparatuses may be realized by a CPU and a program analyzed and executed by the CPU or may be realized as hardware using wired logic.

Further, it is possible to realize any of the methods explained in the present embodiments, by causing a computer such as a personal computer or a workstation to execute a program prepared in advance. The program may be distributed via a network such as the Internet. Further, the program may be executed, as being recorded on a non-transitory computer-readable recording medium such as a hard disk, a flexible disc (FD), a Compact Disc Read-Only Memory (CD-ROM), a Magneto Optical (MO) disc, a Digital Versatile Disc (DVD), or the like and being read by a computer from the recording medium.

According to at least one aspect of the embodiments described above, it is possible to assist medical doctors in making proper diagnoses.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing system comprising processing circuitry configured:
to detect an abnormal site of a subject on a basis of an image of the subject;
to extract a disease candidate on a basis of the abnormal site;
to specify a query for performing a diagnosing process on the disease candidate; and
to present the query.

2. The medical information processing system according to claim 1, wherein the processing circuitry is configured to extract the disease candidate, on a basis of the abnormal site and a medical department with which the subject was registered at a reception.

3. The medical information processing system according to claim 1, wherein the processing circuitry is configured to present the query to a medical doctor.

4. The medical information processing system according to claim 1, wherein the processing circuitry is configured to add the query to a questionnaire to be filled out by the subject.

5. The medical information processing system according to claim 1, wherein the image of the subject is an image taken at a location including at least one selected from between an entrance and a waiting room of a hospital.

6. The medical information processing system according to claim 1, wherein the processing circuitry is configured to detect, as the abnormal site, a site of a body of the subject having an unsatisfactory movement in comparison to a health movement.

7. The medical information processing system according to claim 1, wherein, when presenting the query, the processing circuitry is configured to display a video or a still image taken at a point in time when the abnormal site was imaged.

8. A medical information processing method comprising:
detecting an abnormal site of a subject on a basis of an image of the subject;
extracting a disease candidate on a basis of the abnormal site;
specifying a query for performing a diagnosing process on the disease candidate; and
presenting the query.

9. A non-transitory computer-readable recording medium having recorded thereon a program that causes a computer to execute:
detecting an abnormal site of a subject on a basis of an image of the subject;
extracting a disease candidate on a basis of the abnormal site;
specifying a query for performing a diagnosing process on the disease candidate; and
presenting the query.
